(19) United States
(12) Patent Application Publication
(10) Pub. No.: US 2014/0248270 A1
(43) Pub. Date: Sep. 4, 2014

(54) METHOD FOR TOPICAL TREATMENT OF TAR-RESPONSIVE DERMATOLOGICAL DISORDERS

(71) Applicants: Ruey J. YU, Chalfont, PA (US); Eugene J. VAN SCOTT, Abington, PA (US)

(72) Inventors: Ruey J. YU, Chalfont, PA (US); Eugene J. VAN SCOTT, Abington, PA (US)

(21) Appl. No.: 14/276,470

(22) Filed: May 13, 2014

Related U.S. Application Data
(63) Continuation of application No. 12/638,505, filed on Dec. 15, 2009.
Application No. 12/638,505 is a division of application No. 11/680,227, filed on Feb. 28, 2007, now abandoned.
(60) Provisional application No. 60/778,128, filed on Mar. 1, 2006.

(51) Int. Cl. A61K 35/04; A61K 9/00; A61K 47/14; A61K 45/06

(52) U.S. Cl. CPC A61K 35/04; A61K 45/06; A61K 9/0014; A61K 47/14
USPC 424/134.1; 514/183; 514/75; 514/171; 424/142.1; 514/46; 514/34; 514/154; 424/133.1; 424/685

(57) ABSTRACT

A method of treating a tar-responsive dermatological disorder includes topically applying an anhydrous tar composition to skin of a mammal, preferably a human, that is involved with the disorder, the composition including a wax and a therapeutically effective amount of tar for topical treatment of the tar-responsive dermatological disorder, the composition being in liquid form when at a temperature selected from room temperature and a temperature of skin of a mammal upon application of the composition to the skin of the mammal.

### CROSS-REFERENCE TO RELATED APPLICATIONS

This application is a continuation of U.S. patent application Ser. No. 12/638,505, filed Dec. 15, 2009, which is a divisional of U.S. patent application Ser. No. 11/680,227, filed Feb. 28, 2007. Therefore, this application claims the benefit under 35 U.S.C. §119(e) of U.S. Provisional Application No. 60/778,128, filed Mar. 1, 2006. The entire disclosures of all of these applications are hereby incorporated herein by reference.

### BACKGROUND OF THE INVENTION

This application relates to compositions comprising tar and methods of using such compositions for topical treatment of tar-responsive dermatological disorders.

Inflammatory diseases such as psoriasis, eczema and other dermatoses frequently involve disturbed keratinization with scale formation. The causes of most inflammatory dermatoses are unknown, although immunologic and genetic factors appear to be associated with the development of these diseases. Psoriasis is a chronic inflammatory skin disease characterized by persistent erythema and silvery scales, and remains a disfiguring and disabling cutaneous impairment to millions of people. In the United States, the disease affects approximately 2% of the population. Eczema is also a chronic skin disease characterized by persistent intensive itch with erythema and some scales. Because the etiologies of these diseases are unknown, their prevention remains inconceivable, and therapies have been empiric. In psoriasis, photochemotherapy with psoralens plus ultraviolet A radiation and systemic treatments with old drugs or experimental agents provide short term remission of the disease. Such drugs include methotrexate, cyclosporine, retinoids, fumaric acid esters, glucocorticoids, alefacept, efalizumab, etanercept, infliximab, anti-CD4-antibodies, interleukin diphtheria fusion toxin and ascomycin derivatives. Immunosuppressions leading to serious infections, cancers, acute and chronic toxicity on liver, kidney and bones, etc. from the above treatment have shifted the thought and desire for external treatment.

The use of tar for topical treatment of skin diseases dates back many years. Tar is obtained as a by-product by dry distillation of organic materials such as coal or wood in the absence of oxygen. There are three different types of tar: coal tar, wood tar and shale tar used for topical treatment of psoriasis, atopic dermatitis, seborrheic dermatitis, tinea versicolor, vitiligo, pruritus, yeast or dermatophyte infections. The crude coal tar is dark brownish, messy to handle and has an unpleasant odor. Liquor carbonis detergens (LCD) is an alcohol extract of coal tar emulsified with polysorbate 80 (Tween® 80). However, LCD still has objectionable odor and can stain skin and clothing. The therapeutic effects of commercial tar products are variable and inconsistent due to low bioavailability of the active ingredients, and these products can stain skin and clothing.

### BRIEF SUMMARY OF THE INVENTION

One aspect of the invention is a fast-drying composition comprising a wax and a therapeutically effective amount of tar for topical treatment of a tar-responsive dermatological disorder, the composition being in liquid or light gel form when at a temperature selected from room temperature and a temperature of skin of a mammal upon application of the composition to the skin of the mammal. The active tar ingredients of the tar in the liquid or light gel composition penetrate into the skin readily as the solvents evaporate quickly to provide treated sites with reduced or no stickiness and staining. Preferably, the composition further comprises at least one of a nonionic surfactant and a film former.

Another aspect of the invention is a method of treating a dermatological disorder in a mammal comprising topically applying a tar composition comprising a wax and a therapeutically effective amount of tar to skin of the mammal involved in the disorder, the composition being in liquid or light gel form when at a temperature selected from room temperature and skin temperature of the skin of the mammal. Preferably, the mammal is a human.

### DETAILED DESCRIPTION OF THE INVENTION

We have now discovered that a fast-drying tar composition, preferably a coal tar composition in liquid or light gel form when at room temperature or a composition that becomes a liquid or a light gel when it contacts the skin, can provide (a) superior therapeutic effects and (b) minimal staining to the skin and clothing, when the novel liquid or light gel composition comprising tar, preferably coal tar, and a wax is topically applied to involved skin when treating tar-responsive dermatological disorders. Excellent therapeutic results can be achieved with the following liquid or light gel tar compositions and the method of applying the compositions.

Compositions of the present invention can be formulated as cosmetic compositions or cosmetic products for topical treatment or prevention of dermatological indications or can be formulated as pharmaceutical compositions or pharmaceutical products for topical treatment or prevention of dermatological disorders.

As used herein, the terms “treatment,” “treating,” and the like, refer to obtaining a desired pharmacologic, physiologic, dematologic or cosmetic effect. The effect may be prophylactic in terms of completely or partially preventing a condition or disease or disorder or symptom thereof and/or may be therapeutic in terms of a partial or complete cure for a condition or disease or disorder and/or adverse symptom or effect attributable to the condition or disease or disorder. “Treatment,” thus, for example, covers any treatment of a condition or disease in a mammal, particularly in a human, and includes: (a) preventing the condition or disease, disorder or symptom thereof from occurring in a subject which may be predisposed to the condition or disease or disorder but has not yet been diagnosed as having it; (b) inhibiting the condition or disease, disorder or symptom thereof, such as, arresting its development; and (c) relieving, alleviating or ameliorating the condition or disease or disorder or symptom thereof, such as, for example, causing regression of the condition or disease or disorder or symptom thereof.

Tar-responsive dermatological disorders include, without limitation, psoriasis, eczema, atopic dermatitis, seborrheic dermatitis, tinea versicolor, vitiligo, pruritus, yeast and dermatophyte infections.

The term “light gel” as used herein is a relative description and is in contrast to a heavy gel, and refers to a gel which is readily spreadable when topically applied to the skin without a tacky or heavy feeling to the skin. The preferred light gel is one which becomes liquid or partially liquid upon topical application to the skin.

Coal tar or LCD is formulated in a fast-drying liquid or light gel composition containing a wax. Such liquid or light gel tar composition has optimal bioavailability and occlusion for the active ingredients to penetrate into the skin quickly. The liquid or light gel tar composition may be incorporated with or into and applied using a container having a dauber typically attached to the inside of the container cap, a foam applicator, brush pen applicator, or spray can or container. Preferably, the composition is incorporated into and is topically applied to an involved portion of the skin using a dauber, such as a dauber attached to a removable cap or lid of a container for the composition. As the active ingredients penetrate into the involved skin and the solvents evaporate, the treated skin sites are optionally covered with cream, lotion or simply talc powder. The above process can be repeated once or more than once daily until the disorder has been substantially or completely eradicated. By such steps or method of topical treatment, staining of skin and clothing is eliminated or minimized, and the therapeutic efficacy is markedly enhanced.

We have also discovered that the brownish color in tar or LCD can be removed by mixing a tar solution or LCD with activated charcoal at room temperature, and filtering the mixture. The filtrate is a nearly colorless LCD which does not stain skin or clothing.

In one preferred method, tar, LCD or colorless LCD is dissolved in one or more anhydrous solvents selected from ethanol, isopropyl alcohol, cyclomethicone, propylene glycol, butylene glycol, diisopropyl adipate, diethyl tartarate, triethyl citrate, tripropyl citrate, triisopropyl citrate, isopropyl myristate, isopropyl palmitate, ethoxy diglycol, isododecane (Permethyl™ 99A), isohexadecane or isoeicosane. The concentration of crude tar, preferably coal tar solution or LCD, is about 0.1% to about 99%, preferably about 1% to about 30%, and more preferably about 5% to about 20% by weight.

Although a wide range of concentration of LCD can be used in the composition of the present invention, the preferred concentration used for tar-responsive dermatological disorders can be about 1% to about 30% by weight. In practice, the speed of improvement depends on a number of factors which include LCD concentration, formulation, bioavailability of the active ingredients, frequency of application, duration of topical application, severity of the disease or disorder and the subject's characteristics. In general, a preferred concentration of LCD being used in the composition for topical treatment of psoriasis and eczema can be about 15% by weight.

The concentration of the solvent is about 5% to about 95%, preferably about 20% to about 90%, and more preferably about 30% to about 85% by weight.

A wax substance is added to the above solution. The wax can be a liquid or solid wax including one or more of liquid wax dioctyldodecyl dodecanedioate (DIADD), liquid wax diisocetyl dodecanedioate (DICDD), liquid wax octyldodecyl PPG-3 myristyl ether dimer dilinoleate (PolyEFA), liquid wax stearyl/PPG-3 myristyl ether dimer dilinoleate (PolyIPL), liquid wax dioctyldodecyl dimer dilinoleate (DI-EFA), liquid wax diisostearyl adipate (DISA), liquid wax dicetearyl dimer dilinoleate (IPL), cetyl ester wax (synthetic spermaceti), mineral oil, dimethicone, apple peel wax, avocado wax, bayberry wax, beeswax, candelilla wax, carnauba wax, ceresin, jojoba wax, lanolin wax, mink wax, montan wax, orange peel wax, ouricury wax, ozokerite, palm kernel wax, paraffin, polyethylene glycol (PEG)-beeswax, PEG-carnauba, rice wax, shellac wax, spent grain wax, synthetic beeswax, synthetic Japan wax, or other natural or synthetic waxes. The preferred wax is a liquid wax, such as DIADD, DICDD, PolyEFA, PolyIPL, DI-EFA, DISA and/or IPL. The total concentration of the wax in the final composition can be about 1% to about 50%, preferably about 1% to about 25%, and more preferably about 2% to about 10% by weight. Preferably, the above liquid tar composition is packaged in a container including a dauber for easy and convenient delivery or application of the tar liquid to the involved skin.

Optionally, a nonionic surfactant, film former, water, emollient and occlusive agent can be added to the liquid or light gel tar composition to further enhance the therapeutic effects of coal tar and skin conditioning.

The nonionic surfactant can be selected from the following non-limiting examples:

(1) sorbitan fatty acid esters: e.g., sorbitan laurate, sorbitan palmitate, sorbitan oleate, sorbitan sesquioleate, sorbitan isostearate and sorbitan trioleate;

(2) polyoxyethylene derivatives of sorbitan fatty acid esters: e.g., polysorbate 20, polysorbate 21, PEG-80 sorbitan laurate, polysorbate 40, polysorbate 60, polysorbate 61, polysorbate 65, polysorbate 80, polysorbate 81 and polysorbate 85;

(3) polyoxyethylene fatty glycerides: e.g., PEG-25 and PEG-40 hydrogenated castor oil, polyoxyethylene 7 hydrogenated castor oil and polyoxyethylene 40 hydrogenated castor oil;

(4) polyoxyethylene polyol fatty acid esters: e.g., polyoxyethylene 40 sorbitol septaoleate;

(5) polyoxyethylene fatty ethers: e.g., Laureth™-4, Laureth™-23, Oleth™-2, Oleth™-10, etc.

The concentration of the nonionic surfactant in the final composition can be about 1% to about 40%, preferably about 1% to about 25%, and more preferably about 2% to about 15% by weight.

The film former can be selected from the following non-limiting examples:

(1) copolymers of vinylpyrrolidone (PVP) and long-chain alpha-olefins: e.g., butylated

PVP, vinylpyrrolidone (VP)/hexadecene copolymer, VP/eicosene copolymer, tricontanyl PVP;

(2) polyurethanes;

(3) vinylcaprolactam/VP/dimethylaminoethyl methacrylate copolymer;

(4) vinyl acetate (VA)/butyl maleate/isobornyl acrylate copolymer;

(5) vinylcaprolactam/VP/dimethylaminoethyl methacrylate copolymer;

(6) monoethyl esters of the copolymer of methylvinyl ether and maleic anhydride (PVM/MA copolymer);

(7) PVP/vinylcaprolactam/dimethylaminopropyl methacrylamide acrylate;

(8) isobutylene/ethylmaleimide/hydroxyethylmaleimide copolymer;

(9) monoalkyl esters of poly (methyl vinyl ether/maleic acid):
a. ethyl ester of PVM/MA copolymer;
b. butyl ester of PVM/MA copolymer;
c. i sopropyl ester of PVM/MA copolymer;

(10) vinylpyrrolidone/vinyl acetate copolymer;

(11) dimethiconols and dimethiconol-dimethicone copolyol; or

(12) cellulose and cellulose derivatives (cellulose esters and cellulose ethers): e.g., cellulose acetate, cellulose triacetate, nitrocellulose, ethylcellulose, methylcellulose, hydroxypropyl cellulose, carboxymethyl cellulose, hydroxypropyl methyl cellulose, hydroxyethyl methyl cellulose, microcystalline cellulose, etc.

The concentration of the film former can be about 1% to about 30%, preferably about 1% to about 20%, and more preferably about 1% to about 10% by weight.

The emollient and occlusive agents include for example and without limitation: withioleyl lactate, oleyl acetate, oleyl oleate, oleyl arachidate, oleyl erucate, acetylated lanolin, polyglyceryl oleate, propylene glycol oleate, propylene glycol linoleate, octyldodecyl lactate, octyl oleate, decyl oleate or trioleyl citrate. The concentration of water, emollient or occlusive agents can be about 1% to about 30%, preferably about 1% to about 20%, and most preferably about 1% to about10% by weight.

Powdered absorbents or adsorbents usually have a very large surface area to attract and remove excess materials from the skin surface. These absorbents and adsorbents can include one or more of aluminum silicate, aluminum starch octenylsuccinate, amylodextrin, attapulgite, bentonite, calamine, calcium silicate, cellulose, chalk, colloidal oatmeal, corn flour, corn starch, cyclodextrin, dextrin, diatomaceous earth, dimethylimidazolidinone corn starch, dimethyliminodazolidinone rice starch, fuller's earth, glyceryl starch, hectorite, hydrated silica, kaolin, loess, magnesium aluminum silicate, magnesium carbonate, magnesium hydroxide, magnesium oxide, magnesium silicate, magnesium trisilicate, maltodextrin, microcrystalline cellulose, montmorillonite, moroccan lava clay, oat bran, oat flour, oat meal, oat starch, phaseolus angularis bean starch, potassium aluminum polyacrylate, potato starch, pyrophyllite, rice starch, silica, sodium magnesium fluorosilicate, sodium polyacrylate starch, sodium starch octenylsuccinate, talc, wheat powder, wheat starch, wood powder, zeolite, or other natural or synthetic absorbents and adsorbents. The preferred powdered absorbents and adsorbents are talc, starch powder, cellulose powder and oatmeal powder, and more preferred ones are fine powders of talc in a dispenser.

In one embodiment, a liquid or light gel tar composition of the present invention is topically applied to an involved portion of the skin, the active ingredients of coal tar penetrate into the lesions quickly and the solvents evaporate within a few minutes, usually a minute or two. At this time, the treated skin sites can be lightly covered or dusted with a powder, for example, the talc powder. Such simple two-step treatment can substantially eliminate the staining and odor of the coal tar without adversely affecting its therapeutic benefit.

In another embodiment of the invention, the composition can further comprise at least one topically active pharmaceutical or cosmetic agent or at least one separate composition comprising such agent or agents topically administered alternatively for synergetic or synergistic effects. The topical agents can include one or more of hydroxyacids, polyhydroxy acids, polyhydroxy lactones, ketoacids and related compounds; phenyl alpha acyloxyalkanoic acids and derivatives; N-acyl-aldosamines, N-acylamino acids and related N-acyl compounds; N-(phosphonoalkyl)-aminocarbohydrates, N-(phosphonoalkyl)-amino acids and their related N-(phosphonoalkyl)-compounds; local analgesics and anesthetics; anti-acne agents; anti-bacterial agents; anti-yeast agents; anti-fungal agents; anti-viral agents; anti-infective agents; anti-dandruff agents; anti-dermatitis agents; anti-eczema agents; anti-histamine agents; anti-pruritic agents; anti-emetics; anti-motion sickness agents; anti-inflammatory agents; anti-hyperkeratotic agents; antiperspirants; anti-psoriatic agents; anti-rosacea agents; anti-seborrheic agents; hair conditioners and hair treatment agents; anti-aging and anti-wrinkle agents; anti-anxiety agents; anti-convulsant agents; anti-depressant agents; sunblock and sunscreen agents; skin lightening agents; depigmenting agents; astringents; cleansing agents; corn, callus and wart removing agents; skin plumping agents; skin volumizing agents; skin firming agents; matrix metalloproteinase (MMP) inhibitors; topical cardiovascular agents; wound-healing agents; gum disease or oral care agents; amino acids; peptides; dipeptides; tripeptides; glutathione and its derivatives; oligopeptides; polypeptides; carbohydrates; aminocarbohydrates; vitamins; corticosteroids; tanning agents; hormones or retinoids.

For synergetic or synergistic effects, the cosmetic, pharmaceutical and other topically active agents include abacavir, acebutolol, acetaminophen, acetaminosalol, acetazolamide, acetohydroxamic acid, acetylsalicylic acid, N-acylglutathione ethyl ester and other esters, N-acyl proline ethyl ester and other esters, acitretin, aclovate, acrivastine, actiq, acyclovir, adalimumab, adapalene, adefovir dipivoxil, adenosine, albuterol, alefacept, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, p-aminobenzoic acid, aminocaproic acid, aminolevulinic acid, aminosalicylic acid, amiodarone, amitriptyline, amlodipine, amocarzine, amodiaquin, amorolfine, amoxapine, amphetamine, ampicillin, anagrelide, anastrozole, anthralin, apomorphine, aprepitant, arbutin, aripiprazole, ascorbic acid, ascorbyl palmitate, atazanavir, atenolol, atomoxetine, atropine, azathioprine, azelaic acid, azelastine, azithromycin, bacitracin, beclomethasone dipropionate, bemegride, benazepril, benzilic acid, bendroflumethiazide, benzocaine, benzonatate, benzophenone, benzoyl peroxide, benztropine, bepridil, betamethasone dipropionate, betamethasone valerate, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, carbamide peroxide, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clocortolone pivalate, clomiphene, clonidine, clopidogrel, clotrimazole, clozapine, cocaine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, diclofenac, dicyclomine, didanosine, dihydrocodeine, dihydromorphine, diltiazem, 6,8-dimercaptooctanoic acid (dihydrolipoic acid), diphenhydramine, diphenoxylate, dipyridamole, disopyramide, dobutamine, dofetilide, dolasetron, donepezil, dopa esters, dopamide, dopamine, dorzolamide, doxepin, doxorubicin, doxycycline, doxylamine, doxypin, duloxetine, dyclonine, econazole, efalizumab, eflornithine, eletriptan, emtricitabine, enalapril, ephedrine, epinephrine, epinine, epirubicin, eptifibatide, ergotamine, erythromycin, escitalopram, esmolol, esomeprazole, estazolam, estradiol, etanercept, ethacrynic acid, ethinyl estradiol, ethyl pyruvate, etidocaine, etomidate, famciclovir, famotidine, felodipine, fentanyl, ferulic acid, fexofenadine, flecainide, fluconazole, flucytosine, fluocinolone acetonide, fluocinonide, 5-fluorouracil, fluoxetine, fluphenazine, flurazepam, fluticasone propionate, fluvoxamine, formoterol, furosemide, galactarolactone, galactonic acid, galactonolactone, galantamine, gatifloxacin, gefitinib, gemcitabine, gemifloxacin, glucarolactone, gluconic acid, gluconolactone, glucuronic acid, glucuronolactone, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydrogen peroxide, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, infliximab, irbesartan, irinotecan, isoetharine, isoproterenol, itraconazole, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofloxacin, lidocaine, linezolid, lobeline, loratadine, loperamide, losartan, loxapine, lysergic diethylamide, mafenide, malic acid, maltobionic acid, mandelic acid, maprotiline, mebendazole, mecamylamine, meclizine, meclocycline, memantine, menthol, meperidine, mepivacaine, mequinol, mercaptopurine, mescaline, metanephrine, metaproterenol, metaraminol, metformin, methadone, methamphetamine, methotrexate, methoxamine, methyldopa esters, methyldopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nitrofurantoin, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate O, palonosetron, pantothenic acid, pantoyl lactone, paroxetine, pemoline, penciclovir, penicillamine, penicillins, pentazocine, pentobarbital, pentostatin, pentoxifylline, pergolide, perindopril, permethrin, phencyclidine, phenelzine, pheniramine, phenmetrazine, phenobarbital, phenol, phenoxybenzamine, phentolamine, phenylephrine, phenylpropanolamine, phenytoin, N-(phosphonomethyl)-glycine, N-(phosphonomethyl)-creatine, N-(phosphonomethyl)-tyramine, physostigmine, pilocarpine, pimecrolimus, pimozide, pindolol, pioglitazone, pipamazine, piperonyl butoxide, pirenzepine, podofilox, podophyllin, povidone iodine, pramipexole, pramoxine, prazosin, prednisone, prenalterol, prilocaine, procainamide, procaine, procarbazine, praline, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salicylic acid, salmeterol, scopolamine, selegiline, selenium sulfide, serotonin, sertaconazole, sertindole, sertraline, shale tar, sibutramine, sildenafil, sotalol, streptomycin, strychnine, sulconazole, sulfacetamide, sulfabenz, sulfabenzamide, sulfabromomethazine, sulfacetamide (sodium sulfacetamide), sulfachlorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, sulfur, tacrolimus, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, thalidomide, theobromine, theophylline, thiabendazole, thioctic acid (lipoic acid), thioridazine, thiothixene, thymol, tiagabine, timolol, tinidazole, tioconazole, tirofiban, tizanidine, tobramycin, tocainide, tolazoline, tolbutamide, tolnaftate, tolterodine, tramadol, tranylcypromine, trazodone, triamcinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, wood tar, xanthine, zafirlukast, zaleplon, zinc pyrithione, ziprasidone, zolmitriptan or zolpidem.

General Preparations

Commercially available crude coal tar is a dark viscous paste with a characteristic naphthalene-like odor. The crude tar is slightly soluble in water but is fairly soluble in ethanol and other lipid solvents. A purified coal tar is an alcohol extract of the crude coal tar emulsified with polysorbate 80 (Tween® 80), and is called liquor carbonis detergens, known as LCD or coal tar solution. The commercially available LCD or coal tar solution is a yellow-brownish liquid which still has naphthalene-like odor and can still stain skin and clothing.

Optionally, the color of the coal tar solution can be removed as follows. The following is a typical process to remove the color. Coal tar solution or LCD (USP), 76 g (100 ml) was mixed with 10 g activated charcoal (decolorizing charcoal) and stirred at room temperature for 30 minutes. The mixture was filtered, and the charcoal was washed with 20 ml ethanol. The combined filtrate and the washing (light yellow) were again mixed with 10 g activated charcoal and stirred for 30 minutes. The mixture was filtered and the filtrate was nearly a colorless clear solution which did not stain skin or clothes, but still had coal tar odor.

To prepare a liquid or light gel composition of the present invention, a crude coal tar, preferably coal tar solution or LCD, is dissolved in anhydrous solvents such as ethanol, isopropyl alcohol, propylene glycol, cyclomethicone, triethyl citrate, tripropyl citrate, triisopropyl citrate, diethyl tartarate or polyoxyethylene oleyl ether. The concentration of a crude coal tar, preferably coal tar solution or LCD can be about 0.1% to about 99%, preferably about 1% to about 30%, and more preferably about 5% to about 20% by weight. The total concentration of the solvents can be about 5% to about 95%, with a preferred range of about 20% to about 90%, and more preferably about 30% to about 85%, all by weight.

To prepare a light gel composition, any cosmetically or pharmaceutically acceptable gelling agent is added to the above liquid or light gel composition. Suitable exemplary gelling agents include chitosan, methyl cellulose, ethyl cellulose, polyvinyl alcohol, polyquaterniums, hydroxyethylcellulose, hydroxypropylcellulose, hydroxypropylmethylcellulose, carbomer and ammoniated glycyrrhizinate. The concentration of the gelling agent can be about 0.1% to about 5%, however, the preferred amount is about 0.1% to about 0.5% by weight of the total composition depending on the kind of gelling agent used. As aforementioned, the term “light gel” as used herein is a relative description and is in contrast to a heavy gel, and refers to a gel which is readily spreadable when topically applied to the skin without a tacky or heavy feeling to the skin. The preferred light gel is one which becomes liquid or partially liquid upon topical application to the skin.

A wax substance, preferably liquid wax, such as DIADD, DICDD, liquid wax PolyEFA, liquid wax PolyIPL, liquid wax DI-EFA, liquid wax DISA and/or liquid wax IPL, is added to the above solution. The concentration of the wax can be about 1% to about 50%, preferably about 1% to about 25%, and more preferably about 2% to about 10% by weight.

Optionally, a nonionic surfactant, film former, water, emollient and/or occlusive agent can be added to the liquid or light gel tar composition to further enhance the therapeutic effects of coal tar. The nonionic surfactants include for example, polysorbate 80, polyoxyethylene 40 sorbitol septaoleate and Laureth™-4. The total concentration of the nonionic surfactant may be about 1% to about 40%, preferably about 1% to about 25%, and more preferably about 2% to about 15% by weight.

The film former may include, for example, butylated PVP and VP/hexadecene copolymer. The total concentration of the film formers is about 1% to about 30%, preferably from about 1% to about 20%, and more preferably about 1% to about 10% by weight.

The emollient and occlusive agents may include, for example, oleyl lactate, oleyl acetate, oleyl oleate, oleyl arachidate, oleyl erucate, acetylated lanolin, polyglyceryl oleate, propylene glycol oleate, propylene glycol linoleate, octyldodecyl lactate, octyl oleate, decyl oleate and trioleyl citrate. The concentration of water, emollient or occlusive agents can be about 1% to about 30%, preferably about 1% to about 20%, and more preferably about 1% to about 10% by weight.

Powdered absorbents or adsorbents can be selected from talc, starch powder and cellulose powder. However, the most preferred one is fine powdered talc in a dispenser.

For synergetic or synergistic effects, one or more of a cosmetic, pharmaceutical or other topically active agent can be added into the above liquid or light gel composition of the present invention. The above liquid or light gel tar composition can be packaged in any cosmetically or pharmaceutically acceptable dispenser suitable for topical delivery of a liquid or a light gel to human skin. Examples of such dispensers include spray cans, containers having daubers typically attached to the inside of the container caps, foam applicators, brush pen applicators and ball pens. The preferred one is a container having a dauber for easy and convenient delivery or application of the tar liquid or light gel to the involved skin. Other forms of compositions for delivery of active ingredients of the present invention may be readily blended, prepared or formulated by those skilled in the art in view of the present disclosure.

In one embodiment, the liquid or light gel tar of the present invention is topically applied to involved skin, the active ingredients rapidly penetrate into the lesions and the solvents evaporate within a few minutes, usually a minute or two. At this time, the treated skin sites are optionally covered lightly or dusted with for example, the talc powder. Such simple procedure of topical applications can effectively eliminate odor of coal tar and staining of clothe.

As aforementioned, psoriasis is a chronic inflammatory skin disease characterized by persistent erythema and silvery scales, and remains a disfiguring and disabling cutaneous impairment to millions of people. The prevalence of psoriasis in general population is between 0.4% to 4.8%, with highest incidence in North America and Europe. In U.S. the prevalence is about 2%, and approximately 8 million people have psoriasis.

The involved skin in psoriasis is hyperplastic (thickened), erythematous (red or inflamed), and has thick adherent silvery scales. The degree of thickening is such that lesions are elevated up to 1 mm above the surface of adjacent normal skin; erythema is usually an intense red; the thickened adherent silvery scales cause the surface of involved skin to be markedly rough and uneven. These three attributes of thickness, color and texture can be quantified to allow objective measurement of degree of improvement based on the topical application of the coal tar composition of the present invention.

Degree of Improvement

| | | | Moderate | Substantial | Complete |
|---|---|---|---|---|---|
| | None (0) | Mild (1+) | (2+) | (3+) | (4+) |
| Thick- | Highly | Detectable | Readily | Barely | Normal |
| ness | Elevated | Reduction | Apparent | Elevated | Thickness |
| Texture | Visibly | Palpably | Uneven but | Slightly | Visibly and |
| | Rough | Rough | not Rough | Uneven | Palpably |
| | | | | | Smooth |
| Color | Intense | Red | Dark Pink | Light Pink | Normal Skin |
| | Red | | | | Color |

By means of such parameters, degree of improvement in psoriatic lesions from topical treatment with the coal tar composition of the present invention can be numerically recorded and comparisons made of one treated site to another.

For other forms of dermatoses, such as eczema and seborrheic dermatitis, similar kinds of parameters can be used to determine the efficacy of a topically applied composition containing coal tar.

Embodiments of the invention will now be described further with reference to the following specific, non-limiting examples. Although a wide range of concentration of LCD can be used in the composition of the present invention, and a preferred concentration used for psoriasis and eczema is about 1% to about 30% by weight. We have discovered that the speed of improvement depends on a number of factors which include LCD concentration, formulation, bioavailability of the active ingredients, frequency of application, duration of topical application, severity of the disease or disorder and the subject's characteristics. We have found that a more preferred concentration of LCD which can be used in the composition for topical treatment of psoriasis and eczema can be about 15% by weight, if one concentration is selected for commercial purposes, since this concentration provides good results over a variety of the aforementioned factors.

### EXAMPLE 1

A typical liquid tar composition was formulated as follows. Coal tar solution (LCD, USP) 15 g, was dissolved in anhydrous ethanol 42 g, propylene glycol 5 g, cyclomethicone (DC 345) 15 g, triethyl citrate 5 g, and polyoxyethylene (2) oleyl ether (Brij 93) 10 g. Liquid wax DIADD (dioctyldodecyl dodecanedioate) 5 g, was added to the above solution with stirring. An optional fragrance 3 g was added to the above solution. The liquid tar composition thus formulated contained 15% coal tar and 5% liquid wax in a fast-drying anhydrous vehicle, and was packaged in a container including a dauber for easy application.

### EXAMPLE 2

A typical decolorizing process for coal tar solution was carried out as follows. Coal tar solution (LCD, USP) 38 g (50 ml), was stirred and mixed with activated charcoal 5 g, at room temperature for 30 minutes, and the mixture was filtered. The charcoal was washed with ethanol 10 ml. The combined filtrates are almost colorless and contained active ingredients of the coal tar solution.

### EXAMPLE 3

A male subject, age 45, having plaque psoriasis, topically applied twice daily a 15% liquid tar composition containing 5% liquid wax as formulated in Example 1, for four months. At the end of four months, the erythema of the involved skin almost disappeared completely and the skin became smooth without any scales. His psoriasis had 90% improvement as judged by clinical evaluation.

### EXAMPLE 4

A female subject, age 42, having plaque psoriasis, topically applied twice daily a 15% liquid tar composition containing 5% liquid wax as formulated in Example 1 for two months. At the end of two months, the erythema of the involved skin disappeared completely and the skin became smooth without any scales. Her psoriasis had 100% improvement as judged by clinical evaluation.

### EXAMPLE 5

A female subject, age 81, had plaque psoriasis covering approximately 10% of her body, and the psoriatic lesions had intense red, thin and mild silvery scales. The subject topically applied twice daily a 15% liquid tar composition containing 5% liquid wax as formulated in Example 1 on her right forearm for 14 weeks. At the end of 14 weeks, the intense erythema and silvery scales of her right forearm disappeared completely and the skin became smooth without any scales. Her psoriasis on her right forearm had 100% improvement as judged by clinical evaluation.

### EXAMPLE 6

A female subject, age 50, had psoriasis on her palms and feet, covering approximately 5% of her body, and the psoriatic lesions had red, thick and moderate silvery scales. The subject topically applied twice daily a 15% liquid tar composition containing 5% liquid wax as formulated in Example 1 on both of her feet for 10 weeks. At the end of 10 weeks, the erythema and silvery scales of both of her feet disappeared almost completely and the treated skin became thin without any scales. Her psoriasis on both of her feet had 50% improvement as judged by clinical evaluation.

### EXAMPLE 7

A male subject, age 80, had psoriasis covering approximately 5% of his body, and the psoriatic lesions had red, moderately thick and silvery scales. The subject topically applied twice daily a 15% liquid tar composition containing 5% liquid wax as formulated in Example 1 on his sacral area of psoriatic skin for 6 weeks. At the end of 6 weeks, the erythema and silvery scales of his psoriatic skin disappeared almost completely and the treated skin became thin without any scales. His psoriasis on his treated buttocks had 80% improvement as judged by clinical evaluation.

### EXAMPLE 8

A female subject, age 79, had psoriasis on her feet, covering approximately 2% of her body, and the psoriatic lesions had intense red, moderately thick and silvery scales. The subject topically applied twice daily a 15% liquid tar composition containing 5% liquid wax as formulated in Example 1 on the lateral sides of her feet for 14 weeks. In each topical application, when the liquid tar composition evaporated she also applied an oil-in-water cream on the treated area of the skin. At the end of 14 weeks, the erythema and silvery scales of her treated feet disappeared almost completely and the treated skin became flat without any scales. The psoriasis on her treated feet had 90% improvement as judged by clinical evaluation.

### EXAMPLE 9

A male subject, age 86, had psoriasis covering approximately 10% of his body, and the psoriatic lesions had red, moderately thick and silvery scales. The subject topically applied twice daily a 15% liquid tar composition containing 5% liquid wax as formulated in Example 1 on his psoriatic skin for 18 months. In each topical application, when the liquid tar composition evaporated he also applied an oil-in-water cream on the treated area of the skin. At the end of 18 months, the erythema and silvery scales of his psoriatic skin disappeared completely and the treated skin became normal without any erythema and scales. His psoriasis had 100% improvement as judged by clinical evaluation.

### EXAMPLE 10

A male subject, age 26, had psoriasis on his scalp, ears, neck and other areas of skin, covering approximately 10% of his body, and the psoriatic lesions had red, moderately thick and silvery scales. The subject topically applied twice daily a 15% liquid tar composition containing 5% liquid wax as formulated in Example 1 on his psoriasis for 8 weeks. In each topical application, when the liquid tar composition evaporated he also applied an oil-in-water cream on the treated area of the skin. At the end of 8 weeks, the erythema and silvery scales of his treated scalp, ears and neck disappeared completely and the treated skin became normal without any scales. The psoriasis on the treated scalp, ears and neck had 100% improvement, and the rest of his body had 50% improvement as judged by clinical evaluation.

### EXAMPLE 11

A male subject, age 41, had psoriasis covering approximately 10% of his body, and the psoriatic lesions had red, moderately thick and silvery scales. The subject topically applied twice daily a 15% liquid tar composition containing 5% liquid wax as formulated in Example 1 on his psoriatic skin for 12 months. In each topical application, when the liquid tar composition evaporated he also applied an oil-in-water cream or talc powder on the treated area of the skin. At the end of 12 months, the erythema and silvery scales of his psoriatic skin disappeared almost completely and the treated skin became almost normal without any scales. His psoriasis had 90% improvement as judged by clinical evaluation.

### EXAMPLE 12

A male subject, age 40, had psoriasis covering approximately 10% of his body, and the psoriatic lesions had red, moderately thick and silvery scales. The subject topically applied twice daily a 15% liquid tar composition containing 5% liquid wax as formulated in Example 1 on his psoriasis for 24 months. In each topical application, when the liquid tar composition evaporated he also applied an oil-in-water cream and/or talc powder on the treated area of the skin. At the end of 24 months, the erythema and silvery scales of his treated sites almost disappeared completely and the treated skin became almost normal without any scales. The psoriasis had 90% improvement as judged by clinical evaluation.

### EXAMPLE 13

A female subject, age 39, had psoriasis covering approximately 6% of her body, and the psoriatic lesions had red, moderately thick and silvery scales. The subject topically applied twice daily a 15% liquid tar composition containing 5% liquid wax as formulated in Example 1 on her psoriatic skin for 6 months. In each topical application, when the liquid tar composition evaporated she also applied an oil-in-water cream and/or talc powder on the treated area of the skin. At the end of 6 months, the erythema and silvery scales of her psoriatic skin disappeared completely and the treated skin became normal without any erythema and scales. Her psoriasis had 100% improvement as judged by clinical evaluation.

### EXAMPLE 14

A female subject, age 67, had psoriasis covering approximately 10% of her body, and the psoriatic lesions had red, moderately thick and silvery scales. The subject topically applied twice daily a 15% liquid tar composition containing 5% liquid wax as formulated in Example 1 on her psoriasis for 24 months. In each topical application, when the liquid tar composition evaporated she also applied an oil-in-water cream and/or talc powder on the treated area of the skin. At the end of 24 months, the erythema and silvery scales of her treated sites disappeared completely and the treated skin became normal without any erythema and scales. The psoriasis had 100% improvement as judged by clinical evaluation.

### EXAMPLE 15

A female subject, age 41, had psoriasis covering approximately 10% of her body, and the psoriatic lesions had red, moderately thick and silvery scales. The subject topically applied twice daily a 15% liquid tar composition containing 5% liquid wax as formulated in Example 1 on her psoriatic skin for 5 months. In each topical application, when the liquid tar composition evaporated she also applied an oil-in-water cream and/or talc powder on the treated area of the skin. At the end of 5 months, the erythema and silvery scales of her psoriatic skin disappeared almost completely and the treated skin became nearly normal without any scales. Her psoriasis had 90% improvement as judged by clinical evaluation.

### EXAMPLE 16

A male subject, age 41, had psoriasis covering approximately 30% of his body, and the psoriatic lesions had red, moderately thick and silvery scales. The subject topically applied once daily a 15% liquid tar composition containing 5% liquid wax as formulated in Example 1 on his psoriasis for 7 months. In each topical application, as the liquid tar composition evaporated he also applied an oil-in-water cream and/or talc powder on the treated area of the skin. At the end of 7 months, the erythema and silvery scales of his treated sites improved substantially and the treated skin had 50% improvement as judged by clinical evaluation.

### EXAMPLE 17

A female subject, age 42, had psoriasis covering approximately 10% of her body, and the psoriatic lesions had red, moderately thick and silvery scales. The subject topically applied twice daily a 15% liquid tar composition containing 5% liquid wax as formulated in Example 1 on her psoriasis for 2 months. In each topical application, as the liquid tar composition evaporated she also applied an oil-in-water cream and/or talc powder on the treated area of the skin. At the end of 2 months, the erythema and silvery scales of her treated sites disappeared completely and the treated skin became normal without any erythema and scales. The psoriasis had 100% improvement as judged by clinical evaluation.

### EXAMPLE 18

A female subject, age 47, had psoriasis covering approximately 20% of her body, and the psoriatic lesions had red, moderately thick and silvery scales. The subject topically applied twice daily a 15% liquid tar composition containing 5% liquid wax as formulated in Example 1 on her psoriatic skin for 3 months. In each topical application, as the liquid tar composition evaporated she also applied an oil-in-water cream and/or talc powder on the treated area of the skin. At the end of 3 months, the erythema and silvery scales of her psoriatic skin disappeared completely and the treated skin became normal without any scales. Her psoriasis had 100% improvement as judged by clinical evaluation.

### EXAMPLE 19

A male subject, age 39, had psoriasis covering approximately 10% of his body, and the psoriatic lesions had red, moderately thick and silvery scales. The subject topically applied twice daily a 15% liquid tar composition containing 5% liquid wax as formulated in Example 1 on his psoriasis for 4 months. In each topical application, as the liquid tar composition evaporated he also applied an oil-in-water cream and/or talc powder on the treated area of the skin. At the end of 4 months, the erythema of his treated sites disappeared almost completely and the treated skin became nearly normal without any scales, and the treated skin had 90% improvement as judged by clinical evaluation

### EXAMPLE 20

A male subject, age 45, had psoriasis covering approximately 30% of his body, and the psoriatic lesions had red, moderately thick and silvery scales. The subject topically applied once daily a 15% liquid tar composition containing 5% liquid wax as formulated in Example 1 on his psoriatic skin for 4 months. In each topical application, as the liquid tar composition evaporated he also applied an oil-in-water cream and/or talc powder on the treated area of the skin. At the end of 4 months, the erythema and silvery scales of his psoriatic skin improved substantially, and his psoriasis had 50% improvement as judged by clinical evaluation.

### EXAMPLE 21

A male subject, age 33, had psoriasis covering approximately 10% of his body, and the psoriatic lesions had red, moderately thick and silvery scales. The subject topically applied twice daily a 15% liquid tar composition containing 5% liquid wax as formulated in Example 1 on his psoriasis for 8 months. In each topical application, as the liquid tar composition evaporated he also applied an oil-in-water cream and/or talc powder on the treated area of the skin. At the end of 8 months, the erythema and scales improved moderately, and the treated skin had 25% improvement as judged by clinical evaluation.

### EXAMPLE 22

A male subject, age 46, had psoriasis covering approximately 10% of his body, and the psoriatic lesions had red, moderately thick and silvery scales. The subject topically applied twice daily a 15% liquid tar composition containing 5% liquid wax as formulated in Example 1 on his psoriatic skin for 3 months. In each topical application, as the liquid tar composition evaporated he also applied an oil-in-water cream and/or talc powder on the treated area of the skin. At the end of 3 months, the erythema and silvery scales of his psoriatic skin disappeared almost completely, and the treated skin became nearly normal without any scales. His treated skin had 95% improvement as judged by clinical evaluation.

### EXAMPLE 23

A male subject, age 53, had psoriasis covering approximately 10% of his body, and the psoriatic lesions had red, moderately thick and silvery scales. The subject topically applied twice daily a 15% liquid tar composition containing 5% liquid wax as formulated in Example 1 on his psoriatic skin for 5 months. In each topical application, as the liquid tar composition evaporated he also applied an oil-in-water cream and/or talc powder on the treated area of the skin. At the end of 5 months, the erythema and silvery scales of his psoriatic skin disappeared almost completely, and the treated skin became nearly normal without any scales. His treated skin had 90% improvement as judged by clinical evaluation.

### EXAMPLE 24

A male subject, age 45, had psoriasis covering approximately 10% of his body, and the psoriatic lesions had red, moderately thick and silvery scales. The subject topically applied twice daily a 15% liquid tar composition containing 5% liquid wax as formulated in Example 1 on his psoriatic skin for 4 months. In each topical application, as the liquid tar composition evaporated he also applied an oil-in-water cream and/or talc powder on the treated area of the skin. At the end of 4 months, the erythema and silvery scales of his psoriatic skin disappeared almost completely, and the treated skin became nearly normal without any scales. His treated skin had 90% improvement as judged by clinical evaluation.

### EXAMPLE 25

A male subject, age 89, had psoriasis covering approximately 10% of his body, and the psoriatic lesions had red, moderately thick and silvery scales. The subject topically applied twice daily a 15% liquid tar composition containing 5% liquid wax as formulated in Example 1 on his psoriatic skin for 6 months. In each topical application, as the liquid tar composition evaporated he also applied an oil-in-water cream and/or talc powder on the treated area of the skin. At the end of 6 months, the erythema and silvery scales of his psoriatic skin disappeared almost completely, and the treated skin became nearly normal without any scales. His treated skin had 95% improvement as judged by clinical evaluation.

### EXAMPLE 26

A male subject, age 50, had psoriasis covering approximately 30% of his body, and the psoriatic lesions had red, moderately thick and silvery scales. The subject topically applied twice daily a 15% liquid tar composition containing 5% liquid wax as formulated in Example 1 on his psoriatic skin for one month. In each topical application, as the liquid tar composition evaporated he also applied an oil-in-water cream and/or talc powder on the treated area of the skin. At the end of one month, the erythema and silvery scales of his psoriatic skin improved moderately, and his treated skin had 25% improvement as judged by clinical evaluation.

### EXAMPLE 27

A female subject, age 89, had psoriasis covering approximately 10% of her body, and the psoriatic lesions had red, moderately thick and silvery scales. The subject topically applied occasionally a 15% liquid tar composition containing 5% liquid wax as formulated in Example 1 on her psoriatic skin for 24 months. In each topical application, as the liquid tar composition evaporated she also applied an oil-in-water cream and/or talc powder on the treated area of the skin. At the end of 24 months, the erythema and silvery scales of her psoriatic skin improved substantially, and her treated skin had 50% improvement as judged by clinical evaluation.

### EXAMPLE 28

A typical light gel tar composition was formulated as follows. Coal tar solution (LCD, USP) 15 g, was mixed with propylene glycol 5 g, cyclomethicone (DC345) 10 g, triethyl citrate 5 g, polyoxyethylene (2) oleyl ether (Brij 93) 10 g, dehydrated ethanol 31.8 g, liquiwax DIADD (dioctyldodecyl dodecanedioate) 5 g, purified water 5 g, and oleyl lactate 10 g. Ethylcellulose 0.2 g was added into the above solution with stirring as a gelling agent. An optional fragrance 3 g, was added to the light gel. The light gel tar composition thus formulated contained 15% coal tar and 5% liquid wax.

### EXAMPLE 29

A light gel tar composition was formulated as follows. Coal tar solution (LCD, USP) 15 g, was mixed with propylene glycol 5 g, cyclomethicone (DC345) 10 g, triethyl citrate 5 g, polyoxyethylene (2) oleyl ether (Brij 93) 10 g, dehydrated ethanol 31.9 g, liquiwax DIADD (dioctyldodecyl dodecanedioate) 5 g, purified water 5 g, and oleyl lactate 10 g. Butyl ester of PVM/MA copolymer 0.1 g, was added into the above solution with stirring as a gelling agent. An optional fragrance 3 g, was added to the above light gel. The light gel tar composition thus formulated contained 15% coal tar and 5% liquid wax.

### EXAMPLE 30

A light gel tar composition was formulated as follows. Coal tar solution (LCD, USP) 15 g, was mixed with propylene glycol 5 g, cyclomethicone (DC345) 10 g, triethyl citrate 5 g, polyoxyethylene (2) oleyl ether (Brij 93) 10 g, dehydrated ethanol 27 g, liquiwax DIADD (dioctyldodecyl dodecanedioate) 5 g, purified water 5 g, oleyl lactate 10 g. Ethylcellulose 5 g, was added into the above solution with stirring as a gelling agent. An optional fragrance 3 g, was added to the above light gel. The light gel tar composition thus formulated contained 15% coal tar and 5% liquid wax.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method of treating a tar-responsive dermatological disorder in a mammal comprising topically applying to skin of the mammal involved in the disorder an anhydrous tar composition comprising a liquid wax and a therapeutically effective amount of tar in an anhydrous solvent, the composition being in liquid form at room temperature, and the liquid wax being selected from the group consisting of at least one of dioctyldodecyl dodecanedioate (DIADD), diisocetyl dodecanedioate (DICDD), octyldodecyl PPG-3 myristyl ether dimer dilinoleate (PolyEFA), stearyl/PPG-3 myristyl ether dimer dilinoleate (PolyIPL), dioctyldodecyl dimer dilinoleate (DI-EFA), diisostearyl adipate (DISA), and dicetearyl dimer dilinoleate (IPL).

2. The method of claim 1, wherein the mammal is a human.

3. The method of claim 2, wherein the tar is a liquid tar solution that has been treated with activated carbon whereby the composition does not stain skin or clothing.

4. The method of claim 1, wherein the tar is selected from the group consisting of coal tar, wood tar and shale tar.

5. The method of claim 1, wherein the tar is coal tar.

6. The method of claim 1, wherein the tar is liquor carbonis detergens.

7. The method of claim 1, wherein the anhydrous solvent is selected from the group consisting of at least one of ethanol, isopropyl alcohol, cyclomethicone, propylene glycol, butylene glycol, diisopropyl adipate, diethyl tartarate, triethyl citrate, tripropyl citrate, triisopropyl citrate, isopropyl myristate, isopropyl palmitate, ethoxy diglycol, isododecane, isohexadecane or isoeicosane.

8. The method of claim 1, wherein the composition is applied to the skin using a dauber.

9. The method of claim 1, wherein the liquid wax is dioctyldodecyl dodecanedioate.

10. The method of claim 1, further comprising at least one of a nonionic surfactant and a film former.

11. The method of claim 10, wherein the nonionic surfactant is selected from the group consisting of at least one of a sorbitan fatty acid ester; a polyoxyethylene derivative of a sorbitan fatty acid ester; a polyoxyethylene fatty glyceride; a polyoxyethylene polyol fatty acid ester; and a polyoxyethylene fatty ether.

12. The method of claim 11, wherein the sorbitan fatty acid ester is selected from the group consisting of sorbitan laurate, sorbitan palmitate, sorbitan oleate, sorbitan sesquioleate, sorbitan isostearate and sorbitan trioleate.

13. The method of claim 11, wherein the polyoxyethylene derivative of a sorbitan fatty acid ester is selected from the group consisting of polysorbate 20, polysorbate 21, PEG-80 sorbitan laurate, polysorbate 40, polysorbate 60, polysorbate 61, polysorbate 65, polysorbate 80, polysorbate 81 and polysorbate 85.

14. The method of claim 11, wherein the polyoxyethylene fatty glyceride is selected from the group consisting of PEG-25 hydrogenated castor oil, PEG-40 hydrogenated castor oil, polyoxyethylene 7 hydrogenated castor oil and polyoxyethylene 40 hydrogenated castor oil.

15. The method of claim 11, wherein the polyoxyethylene polyol fatty acid ester is polyoxyethylene 40 sorbitol septaoleate.

16. The method of claim 10, wherein the film former is selected from the group consisting of at least one copolymer of vinylpyrrolidone (PVP) and a long-chain alpha-olefin, a polyurethane, a vinylcaprolactam/vinylpyrrolidone (VP)/dimethylaminoethyl methacrylate copolymer, a vinyl acetate (VA)/butyl maleate/isobornyl acrylate copolymer, a vinylcaprolactam/VP/dimethylaminoethyl methacrylate copolymer, a monoethyl ester of a copolymer of methylvinyl ether and maleic anhydride (PVM/MA copolymer), a PVP/vinylcaprolactam/dimethylaminopropyl methacrylamide acrylate, an isobutylene/ethylmaleimide/hydroxyethylmaleimide copolymer, a monoalkyl ester of poly (methyl vinyl ether/maleic acid), a vinylpyrrolidone/vinyl acetate copolymer, a dimethiconol, a dimethiconol-dimethicone copolyol, cellulose and a cellulose derivative, wherein the cellulose derivative is selected from the group consisting of cellulose acetate, cellulose triacetate, nitrocellulose, ethylcellulose, methylcellulose, hydroxypropyl cellulose, carboxymethyl cellulose, hydroxypropyl methyl cellulose, hydroxyethyl methyl cellulose and microcystalline cellulose.

17. The method of claim 16, wherein the copolymer of vinylpyrrolidone (PVP) and a long-chain alpha-olefin is selected from the group consisting of butylated PVP, vinylpyrrolidone (VP)/hexadecene copolymer, VP/eicosene copolymer and tricontanyl PVP.

18. The method of claim 16, wherein the monoalkyl ester of poly (methyl vinyl ether/maleic acid) is selected from the group consisting of ethyl ester of PVM/MA copolymer, butyl ester of PVM/MA copolymer and isopropyl ester of PVM/MA copolymer.

19. The method of claim 1, wherein the tar is present in an amount of about 0.1% to about 99%, and the liquid wax is present in an amount of about 1% to about 50%, all amounts being percent by weight.

20. The method of claim 19, further comprising at least one of a nonionic surfactant and a film former, wherein the nonionic surfactant, when present, is present in an amount of about 1% to about 40%, and the film former, when present, is present in an amount of about 1% to about 30%, all amounts being percent by weight.

21. The method of claim 20, wherein the tar is present in an amount of about 1% to about 30%, the liquid wax is present in an amount of about 1% to about 25%, wherein the nonionic surfactant, when present, is present in an amount of about 1% to about 25%, and the film former, when present, is present in an amount of about 1% to about 20%, all amounts being percent by weight.

22. The method of claim 21, wherein the tar is present in an amount of about 5% to about 20%, the liquid wax is present in an amount of about 2% to about 10%, the nonionic surfactant, when present, is present in an amount of about 2% to about 15%, and the film former, when present, is present in an amount of about 1% to about 10%, all amounts being percent by weight.

23. The method of claim 1, wherein the method further comprises topically applying as part of the composition or separately in conjunction with the composition at least one topically active pharmaceutical or cosmetic agent or at least one separate composition comprising at least one topically active pharmaceutical or cosmetic agent.

24. The method of claim 23, wherein the topically active pharmaceutical or cosmetic agent is selected from the group consisting of one or more of a hydroxyacid, polyhydroxy acid, polyhydroxy lactone, ketoacid and related compounds; phenyl alpha acyloxyalkanoic acid and derivatives; N-acyl-aldosamines, N-acylamino acids and related N-acyl compounds; N-(phosphonoalkyl)-aminocarbohydrates, N-(phosphonoalkyl)-amino acids and their related N-(phosphonoalkyl)-compounds; local analgesic, local anesthetic; anti-acne agent; anti-bacterial agent; anti-yeast agent; anti-fungal agent; anti-viral agent; anti-infective agent; anti-dandruff agent; anti-dermatitis agent; anti-eczema agent; anti-histamine agent; anti-pruritic agent; anti-emetic; anti-motion sickness agent; anti-inflammatory agent; anti-hyperkeratotic agent; antiperspirant; anti-psoriatic agent; anti-rosacea agent; anti-seborrheic agent; hair conditioner, hair treatment agent; anti-aging agent, anti-wrinkle agent; anti-anxiety agent; anti-convulsant agent; anti-depressant agent; sunblock agent, sunscreen agent; skin lightening agent; depigmenting agent; astringent; cleansing agent; corn, callus or wart removing agent; skin plumping agent; skin volumizing agent; skin firming agent; matrix metalloproteinase (MMP) inhibitor; topical cardiovascular agent; wound-healing agent; gum disease or oral care agent; amino acid; peptide; dipeptide; tripeptide; glutathione and its derivatives; oligopeptide; polypeptide; carbohydrate; aminocarbohydrate; vitamin; corticosteroid; tanning agent; hormone and retinoid.

25. The method of claim 23, wherein the topically active pharmaceutical or cosmetic agent is selected from the group consisting of one or more of abacavir, acebutolol, acetaminophen, acetaminosalol, acetazolamide, acetohydroxamic acid, acetylsalicylic acid, N-acylglutathione ethyl ester and other esters, N-acyl proline ethyl ester and other esters, acitretin, aclovate, acrivastine, actiq, acyclovir, adalimumab, adapalene, adefovir dipivoxil, adenosine, albuterol, alefacept, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, p-aminobenzoic acid, aminocaproic acid, aminolevulinic acid, aminosalicylic acid, amiodarone, amitriptyline, amlodipine, amocarzine, amodiaquin, amorolfine, amoxapine, amphetamine, ampicillin, anagrelide, anastrozole, apomorphine, aprepitant, arbutin, aripiprazole, ascorbic acid, ascorbyl palmitate, atazanavir, atenolol, atomoxetine, atropine, azathioprine, azelaic acid, azelastine, azithromycin, bacitracin, beclomethasone dipropionate, bemegride, benazepril, benzilic acid, bendroflumethiazide, benzocaine, benzonatate, benzophenone, benzoyl peroxide, benztropine, bepridil, betamethasone dipropionate, betamethasone valerate, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, carbamide peroxide, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clocortolone pivalate, clomiphene, clonidine, clopidogrel, clotrimazole, clozapine, cocaine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, diclofenac, dicyclomine, didanosine, dihydrocodeine, dihydromorphine, diltiazem, 6,8-dimercaptooctanoic acid (dihydrolipoic acid), diphenhydramine, diphenoxylate, dipyridamole, disopyramide, dobutamine, dofetilide, dolasetron, donepezil, dopa esters, dopamide, dopamine, dorzolamide, doxepin, doxorubicin, doxycycline, doxylamine, doxypin, duloxetine, dyclonine, econazole, efalizumab, eflornithine, eletriptan, emtricitabine, enalapril, ephedrine, epinephrine, epinine, epirubicin, eptifibatide, ergotamine, erythromycin, escitalopram, esmolol, esomeprazole, estazolam, estradiol, etanercept, ethacrynic acid, ethinyl estradiol, ethyl pyruvate, etidocaine, etomidate, famciclovir, famotidine, felodipine, fentanyl, ferulic acid, fexofenadine, flecainide, fluconazole, flucytosine, fluocinolone acetonide, fluocinonide, 5-fluorouracil, fluoxetine, fluphenazine, flurazepam, fluticasone propionate, fluvoxamine, formoterol, furosemide, galactarolactone, galactonic acid, galactonolactone, galantamine, gatifloxacin, gefitinib, gemcitabine, gemifloxacin, glucarolactone, gluconic acid, gluconolactone, glucuronic acid, glucuronolactone, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydrogen peroxide, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, infliximab, irbesartan, irinotecan, isoetharine, isoproterenol, itraconazole, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofloxacin, lidocaine, linezolid, lobeline, loratadine, loperamide, losartan, loxapine, lysergic diethylamide, mafenide, malic acid, maltobionic acid, mandelic acid, maprotiline, mebendazole, mecamylamine, meclizine, meclocycline, memantine, menthol, meperidine, mepivacaine, mequinol, mercaptopurine, mescaline, metanephrine, metaproterenol, metaraminol, metformin, methadone, methamphetamine, methotrexate, methoxamine, methyldopa esters, methyldopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nitrofurantoin, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate O, palonosetron, pantothenic acid, pantoyl lactone, paroxetine, pemoline, penciclovir, penicillamine, penicillin, pentazocine, pentobarbital, pentostatin, pentoxifylline, pergolide, perindopril, permethrin, phencyclidine, phenelzine, pheniramine, phenmetrazine, phenobarbital, phenol, phenoxybenzamine, phentolamine, phenylephrine, phenylpropanolamine, phenytoin, N-(phosphonomethyl)-glycine, N-(phosphonomethyl)-creatine, N-(phosphonomethyl)-tyramine, physostigmine, pilocarpine, pimecrolimus, pimozide, pindolol, pioglitazone, pipamazine, piperonyl butoxide, pirenzepine, podofilox, podophyllin, povidone iodine, pramipexole, pramoxine, prazosin, prednisone, prenalterol, prilocaine, procainamide, procaine, procarbazine, praline, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salmeterol, scopolamine, selegiline, selenium sulfide, serotonin, sertaconazole, sertindole, sertraline, shale tar, sibutramine, sildenafil, sotalol, streptomycin, strychnine, sulconazole, sulfacetamide, sulfabenz, sulfabenzamide, sulfabromomethazine, sulfacetamide (sodium sulfacetamide), sulfachlorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, sulfur, tacrolimus, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, thalidomide, theobromine, theophylline, thiabendazole, thioctic acid (lipoic acid), thioridazine, thiothixene, thymol, tiagabine, timolol, tinidazole, tioconazole, tirofiban, tizanidine, tobramycin, tocainide, tolazoline, tolbutamide, tolnaftate, tolterodine, tramadol, tranylcypromine, trazodone, triamcinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, wood tar, xanthine, zafirlukast, zaleplon, zinc pyrithione, ziprasidone, zolmitriptan or zolpidem.

26. The method of claim 1, wherein the method further comprises, after topically applying the composition, topically applying to the area where the composition was applied an absorbent or adsorbent powder.

27. The method of claim 26, wherein the absorbent or adsorbent powder is a powder selected from the group consisting of at least one of aluminum silicate, aluminum starch octenylsuccinate, amylodextrin, attapulgite, bentonite, calamine, calcium silicate, cellulose, chalk, colloidal oatmeal, corn flour, corn starch, cyclodextrin, dextrin, diatomaceous earth, dimethylimidazolidinone corn starch, dimethyliminodazolidinone rice starch, fuller's earth, glyceryl starch, hectorite, hydrated silica, kaolin, loess, magnesium aluminum silicate, magnesium carbonate, magnesium hydroxide, magnesium oxide, magnesium silicate, magnesium trisilicate, maltodextrin, microcrystalline cellulose, montmorillonite, moroccan lava clay, oat bran, oat flour, oat meal, oat starch, phaseolus angularis bean starch, potassium aluminum polyacrylate, potato starch, pyrophyllite, rice starch, silica, sodium magnesium fluorosilicate, sodium polyacrylate starch, sodium starch octenylsuccinate, talc, wheat powder, wheat starch, wood powder and zeolite, or other natural or synthetic absorbents and adsorbents.

28. The method of claim 26, wherein the absorbent or adsorbent powder is a powder selected from the group consisting of at least one of talc, starch powder, cellulose powder and oatmeal powder.

29. The method of claim 1, wherein the composition is applied using a dauber attached to the inside of a cap of a container containing the composition, a foam applicator, a brush pen applicator, or as a spray.

30. The method of claim 1, wherein the dermatological disorder is selected from the group consisting of psoriasis, eczema, atopic dermatitis, seborrheic dermatitis and pruritus.

31. The method of claim 1, wherein the liquid wax selected from the group consisting of at least one of dioctyldodecyl dodecanedioate, diisocetyl dodecanedioate, diisostearyl adipate and dioctyldodecyl dimer dilinoleate, and the composition further comprising at least one non-active ingredient selected from the group consisting of at least one of ethanol, isopropyl alcohol, propylene glycol, butylene glycol, ethoxy diglycol, cyclomethicone, dimethicone, isododecane, isohexadecane, triethyl citrate, diethyl tartarate, diisopropyl adipate, polyoxyethylene (2) oleyl ether, PEG-40 hydrogenated castor oil, polyoxyethylene fatty ether, and polysorbate 20.

32. The method of claim 31, wherein the liquid wax is dioctyldodecyl dodecanedioate and the non-active ingredient is selected from the group consisting of at least one of ethanol, propylene glycol, cyclomethicone, triethyl citrate, and polyoxyethylene (2) oleyl ether.

33. The method of claim 32, wherein the composition is applied from a package having a container including a dauber.

34. The method of claim 31, further comprising at least one topically active pharmaceutical or cosmetic agent or at least one separate composition comprising at least one topically active pharmaceutical or cosmetic agent.

35. The method of claim 34, wherein the topically active pharmaceutical or cosmetic agent is N-acyl proline ethyl ester.

36. The method of claim 31, wherein the dermatological disorder is selected from the group consisting of psoriasis, eczema, atopic dermatitis, seborrheic dermatitis and pruritus.
